(19) United States
(12) Patent Application Publication
(10) Pub. No.: US 2020/0000323 A1
(43) Pub. Date: Jan. 2, 2020

(54) MICRO ROBOTIC IMAGING DEVICE FOR LAPAROSCOPIC SURGERY

(71) Applicant: COVIDIEN LP, Mansfield, MA (US)

(72) Inventor: MATTHEW S. COWLEY, FREDERICK, CO (US)

(21) Appl. No.: 16/404,250

(22) Filed: May 6, 2019

Related U.S. Application Data
(60) Provisional application No. 62/690,611, filed on Jun. 27, 2018.

(51) Int. Cl. A61B 1/00; A61B 1/04; A61B 1/045

(52) U.S. Cl. CPC A61B 1/00156; A61B 1/045; A61B 1/00016; A61B 1/041

(57) ABSTRACT

A micro robotic imaging device includes a housing, a camera coupled to the housing, and legs coupled to the housing. The legs are movable relative to one another to move the camera within a patient's body for capturing imagery of the patient's body.

### CROSS REFERENCE TO RELATED APPLICATION

The present application claims the benefit of and priority to U.S. Provisional Application Ser. No. 62/690,611, filed on Jun. 27, 2018 the entire contents of which are incorporated herein by reference.

### TECHNICAL FIELD

The present disclosure relates to laparoscopic surgery and, more particularly, to robotic imaging for effectuating laparoscopic surgery.

### BACKGROUND

Laparoscopic surgery is a minimally invasive surgical technique performed in the abdomen through multiple small incisions. One of the surgical tools used in laparoscopic surgery is an imaging device such as a laparoscope. A laparoscope conventionally includes a camera that produces a video image of the target area, such as tissue within the body cavity of a patient, for display on a video monitor. The laparoscope can be inserted through a transcutaneous trocar in a small incision to provide visualization while performing surgery in the abdominal cavity. Typically, either a robotic arm or an assistant are dedicated to positioning the laparoscope so that surgeon can see the target area within the body cavity of the patient. Cameras for these imaging devices are often 2D or 3D, but provide a very shallow depth of field, requiring frequent re-positioning of these cameras to obtain all the necessary images of the target area.

### SUMMARY

Accordingly, a continuing need exits to provide imaging devices that provide a broad depth of field and limit positioning challenges occasioned by the robotic arm or the assistant for obtaining such imaging.

According to one aspect, the present disclosure is directed to a micro robotic imaging device. The micro robotic imaging device includes a housing, a camera coupled to the housing, and legs coupled to the housing. The legs are movable relative to one another to move the camera within a patient's body for capturing imagery of the patient's body.

In some embodiments, the legs, the camera, or combinations thereof may be in wireless communication with a remote console. The remote console may be configured to operate the legs, the camera, or combinations thereof.

In certain embodiments, the camera may be movable relative to the housing.

In embodiments, the legs may be movable relative to the housing between a retracted position and an extended position. When the legs are disposed in the retracted position, the micro robotic imaging device may be configured to pass through a surgical cannula for selectively positioning the micro robotic imaging device within the patient's body. When the legs are disposed in the extended position, the legs may be movable relative to the housing to enable the micro robotic imaging device to crawl.

In some embodiments, a first leg of the legs may include a foot configured to grasp tissue. The foot may be movable relative to the first leg. The foot may include a first arm and a second arm.

According to another aspect, the present disclosure is directed to a micro robotic imaging system for capturing imagery of a patient's body cavity. The micro robotic imaging system includes a remote console and a micro robotic imaging device in communication with the remote console. The micro robotic imaging device includes a housing, a camera coupled to the housing, and legs movably coupled to the housing to enable the micro robotic imaging device to crawl through the patient's body cavity.

In certain embodiments, the remote console and the micro robotic imaging device may be wirelessly coupled. The remote console may be actuatable to operate the micro robotic imaging device.

The micro robotic imaging system may further include a surgical cannula, wherein the micro robotic imaging device may be configured to pass through the surgical cannula. The legs may be movable between a retracted position and an extended position such that in the retracted position, the micro robotic imaging device can pass through the surgical cannula, and in the extended position, the micro robotic imaging device cannot pass through the surgical cannula.

The micro robotic imaging system may further include a grasper configured to pass the micro robotic imaging device through the surgical cannula and selectively position the micro robotic imaging device within the patient's body cavity.

In accordance with yet another aspect of the present disclosure, a method of in vivo imaging of a patient's body with a micro robotic imaging system is provided. The method includes advancing a micro robotic imaging device into the patient's body, remotely controlling the micro robotic imaging device so that the micro robotic imaging device crawls through the patient's body, and capturing imagery of the patient's body with the micro robotic imaging device.

The method may include moving legs of the micro robotic imaging device so that the micro robotic imaging device crawls along tissue within the patient's body.

The method may include moving a camera of the micro robotic imaging device to capture the imagery of the patient's body.

The method may include moving the legs of the micro robotic imaging device to enable the micro robotic imaging device to fit through a surgical cannula for selective passage into or out of the patient's body. The surgical cannula may have a diameter through which the micro robotic imaging device passes. The diameter may be about 5 mm to about 15 mm.

Other aspects, features, and advantages will be apparent from the description, the drawings, and the claims that follow.

### BRIEF DESCRIPTION OF DRAWINGS

The accompanying drawings, which are incorporated in and constitute a part of this specification, illustrate embodiments of the disclosure and, together with a general description of the disclosure given above and the detailed description given below, serve to explain the principles of the disclosure, wherein:

FIG. 1 is a front, perspective view of a micro robotic imaging device in an extended position in accordance with the present disclosure;

FIG. 2 is a rear, perspective view of the micro robotic imaging device of FIG. 1;

FIG. 3 is a side, perspective view of the micro robotic imaging device of FIGS. 1 and 2 in a retracted position; and

FIGS. 4-7 are progressive views illustrating the micro robotic imaging device of FIGS. 1-3 being utilized in a laparoscopic surgical procedure.

### DETAILED DESCRIPTION

Embodiments of the presently disclosed micro robotic imaging devices are described in detail with reference to the drawings, in which like reference numerals designate identical or corresponding elements in each of the several views. As commonly known, the term “clinician” refers to a doctor, a nurse, or any other care provider and may include support personnel. Additionally, the term “proximal” refers to the portion of structure that is closer to the clinician and the term “distal” refers to the portion of structure that is farther from the clinician. In addition, directional terms such as front, rear, upper, lower, top, bottom, and the like are used simply for convenience of description and are not intended to limit the disclosure attached hereto.

In the following description, well-known functions or constructions are not described in detail to avoid obscuring the present disclosure in unnecessary detail.

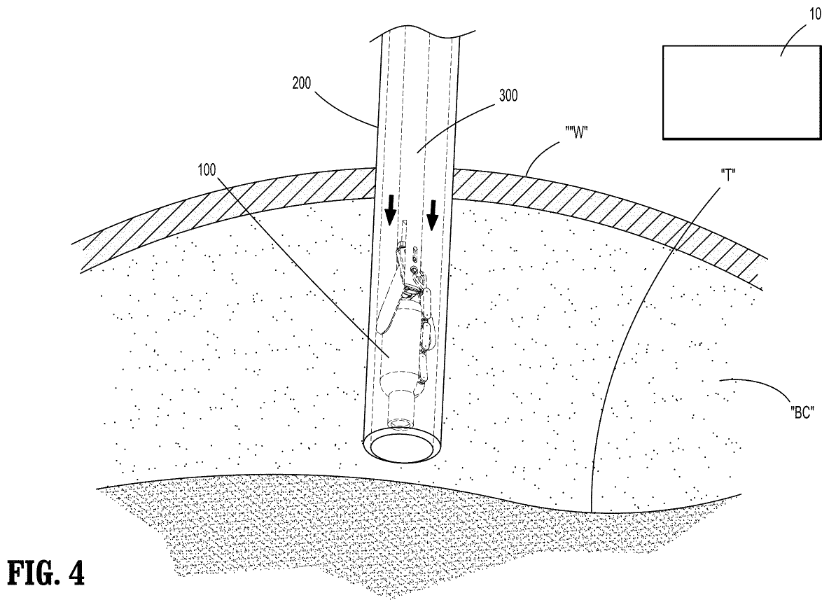

The presently disclosed micro robotic imaging devices are configured to work with robotic surgical systems. Such systems employ various robotic elements to assist the clinician and allow remote operation (or partial remote operation) of surgical instrumentation such as the presently disclosed micro robotic imaging devices, which can be in the form of a micro robotic wireless camera. The robotic surgical systems may be employed with one or more consoles 10 (FIG. 4) that are next to the operating theater or located in a remote location, for communicating with, or operating, such surgical instrumentation wirelessly, for example. As can be appreciated, console 10 may include any number of mechanical and electrical components such as computers, controllers, displays, memory, data, software, etc. so that a highly skilled clinician may perform multiple operations in multiple locations without leaving his/her remote console which can be both economically advantageous and a benefit to the patient or a series of patients.

For a detailed description of exemplary medical work stations and/or components thereof, reference may be made to U.S. Patent Application Publication No. 2012/0116416, and PCT Application Publication No. WO2016/025132, the entire contents of each of which are incorporated by reference herein.

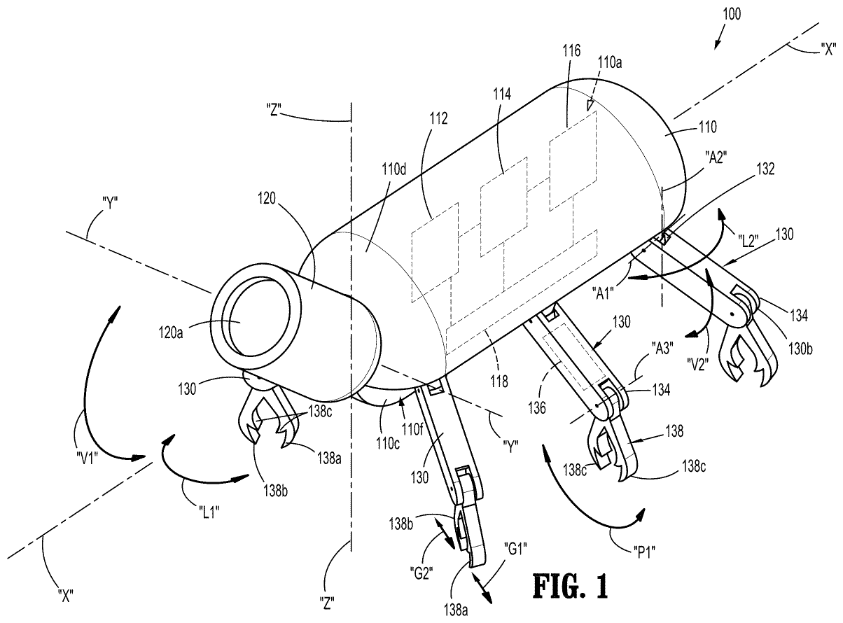

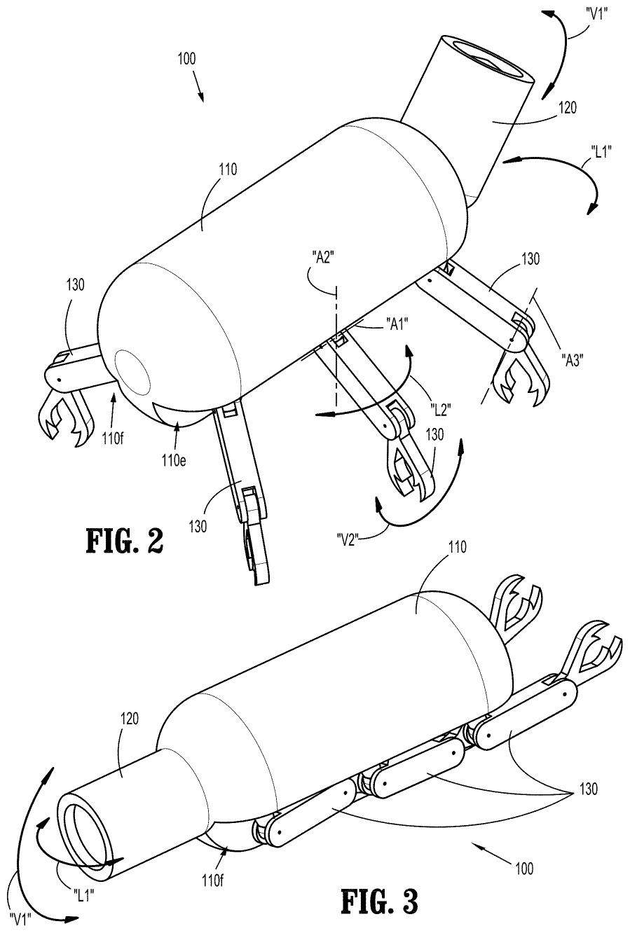

Turning now to FIGS. 1-3, a micro robotic imaging device of the present disclosure is generally referred to as 100. Micro robotic imaging device or MRID 100 includes a housing 110, a camera 120 supported on housing 110, and a plurality of legs 130 pivotally coupled to housing 110. Housing 110 defines a cavity 110a therein that supports any number of motors 112, circuitry 114 (e.g., CPU, sensors, chips, transmitters, receivers, memory, wiring, speakers, microphones, etc.), power supplies 116 (e.g., batteries), and/or mechanical drives 118 (e.g., gears, cables, pulleys, bearings, screws, nuts, etc.) that cooperate to effectuate operation of camera 120 and/or legs 130. Housing 110 further defines a longitudinal axis “X” therethrough and axes “Y” and “Z” on a distal end portion 110d of housing 110, where axes “X,” “Y,” and “Z” are disposed in perpendicular relation to one another so as to define a three dimensional Cartesian coordinate system. Housing 110 further includes a keel 110c that extends along a bottom surface of housing 110 and defines storage channels 110e, 110f (FIG. 2) along opposite sides of keel 110c.

As indicated by vertical arrows “V1” and lateral arrows “L1,” camera 120 of MRID 100 may be supported on housing 110 and polyaxially movable about longitudinal axis “X,” and relative to axes “Y” and “Z” of housing 110, to obtain imaging of a target site captured by camera 120. Camera 120 can be configured to communicate with console 10 (e.g., by wired or wireless communication such as Bluetooth, Wi-Fi, etc.), for instance, to transmit data thereto, or receive data therefrom. Camera 120 is configured to communicate with circuitry 114, such as one or more memory devices thereof (e.g., RAM, ROM, etc.), for example, to store data of imagery and/or audio (e.g., pictures, video, sound, etc.) thereon. Camera 120 includes one or more lens' 120a that receive imagery and cooperate with circuitry 114 of housing 110 to store and/or transmit data of imagery.

Each leg 130 of MRID 100 has a first end 130a that supports a housing joint 132 and a second end 130b that supports a foot joint 134. Housing joint 132 defines axes “A1” and “A2” and couples leg 130 to housing 110 such that leg 130 can polyaxially pivot about axes “A1” and “A2” and relative to housing 110 (e.g., laterally and/or vertically), as indicated by lateral arrows “L2” and vertical arrows “V2.” Leg 130 supports a foot driver 136 that is operably coupled to motors 112, circuitry 114, power supplies 116 and/or mechanical drives 118 supported in housing 110. Foot driver 136 is also operatively coupled to a foot 138 coupled to foot joint 134 of leg 130 that defines pivot axis “A3” for foot 138. Foot driver 136 is actuatable to pivot foot 138 about pivot axis “A3” of foot joint 134, as indicated by arrows “P1.” Foot driver 136 can include one or more suitable mechanical components (e.g., gears, pulleys, cables, screws, etc.) and/or electrical components (e.g., motors, circuitry, etc.) to manipulate foot 138.

Foot 138 of leg 130 includes a first arm 138a and a second arm 138b, each of which include teeth 138c that are configured to facilitate tissue gripping. As illustrated by arrows “G1” and “G2,” foot driver 136 can be configured to actuate foot 138 such that first and/or second arms 138a, 138b thereof pivot about pivot axis “A3” relative to one another between open and closed positions to selectively grasp tissue between first and second arms 138a, 138b. In the open position of foot 138, first and second arms 138a, 138b of foot 138 are spaced apart or unapproximated from one another, and in the closed position (not shown) of foot 138, first and second arms 138a, 138b thereof are approximated or in close proximity to one another (and may be in contact with one another).

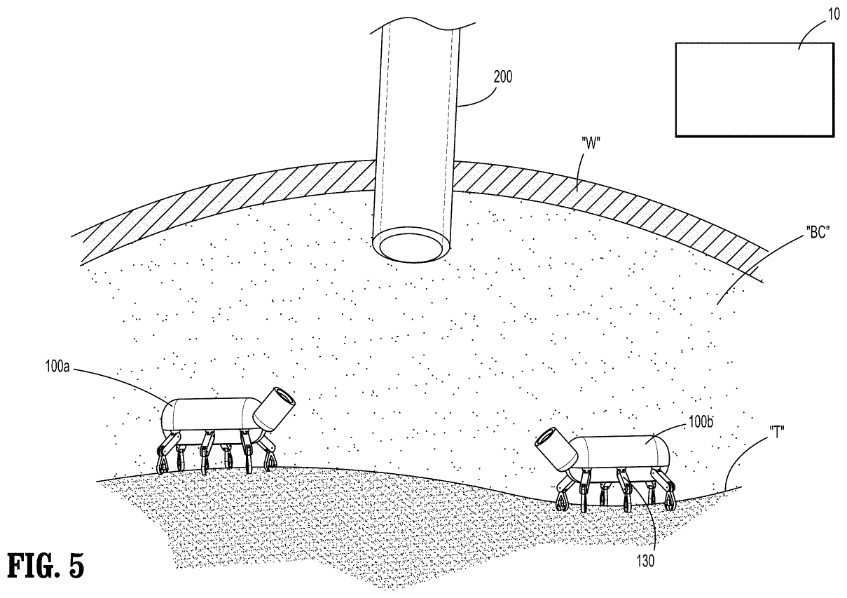

As seen in FIGS. 2 and 3, legs 130 of MRID 100 are movable between an extended position (FIG. 2) and a retracted position (FIG. 3). In the extended position (FIG. 2), legs 130, or portions thereof, are selectively movable, together and/or independent of one another, to enable MRID 100 to crawl and/or climb (ascend and/or descend) along a surface such as tissue “T” (see FIGS. 4 and 5). In the retracted position (FIG. 3), MRID 100 is positioned to be inserted into a body cavity “BC,” and/or removed therefrom, through a cannula 200, such as trocar (e.g., 5 mm-15 mm), by a surgical instrument 300, such as a grasper. For a more detailed description an example trocar, reference can be made to U.S. Pat. No. 6,482,181 to Racenet et al., and for a more detailed description of an example grasper, reference can be made to U.S. Patent Application Publication No. 2017/0224367 to Kapadia, the entire contents of each of which are incorporated by reference herein.

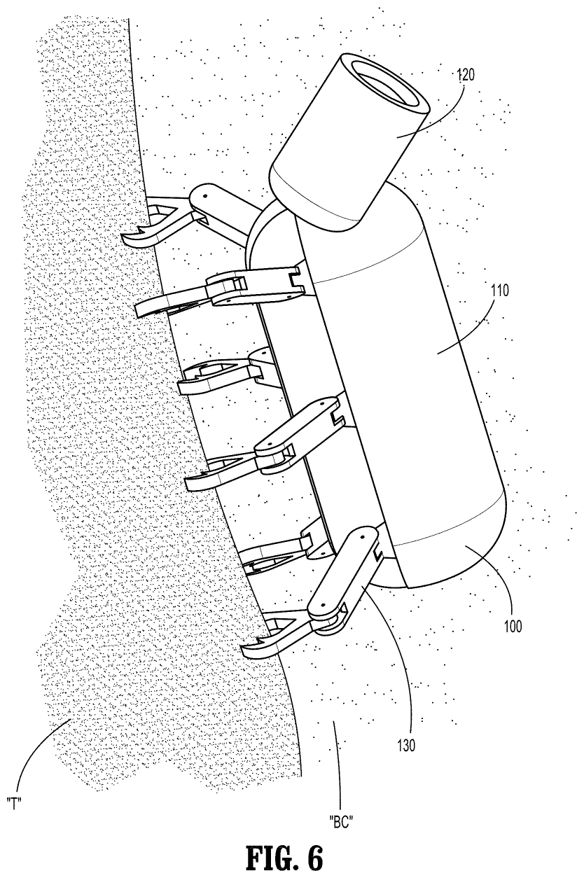

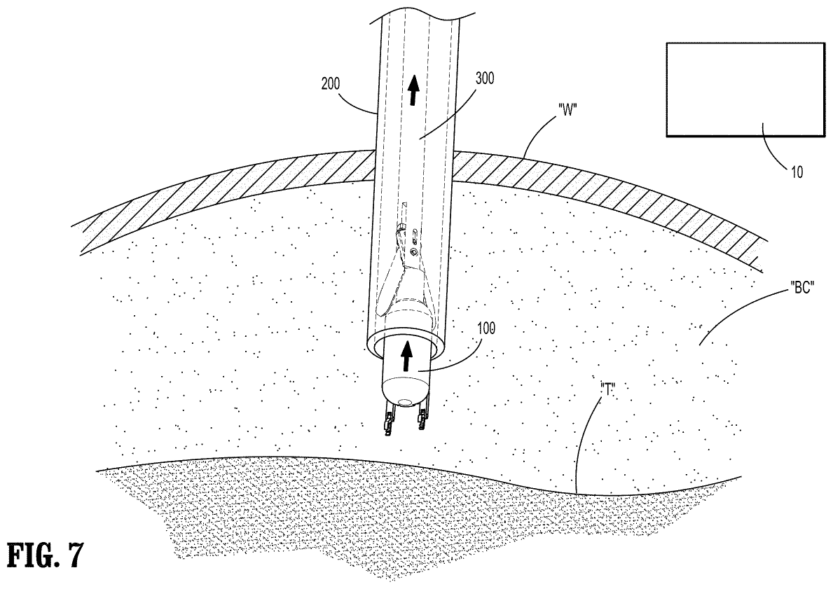

With reference to FIGS. 1-6, with cannula 200 inserted through, for example, an abdominal wall “W,” and MRID 100 disposed in the retracted position thereof, grasper 300 can grasp MRID 100 and advance MRID 100 through cannula 200 and into body cavity “BC.” Any number of MRID's 100, such as MRID 100a and MRID 100b, may be advanced through cannula 200 and positioned into body cavity “BC” where each MRID 100 can be remotely positioned into the extended position thereof to enable crawling movement of MRID 110. When in the extended position, each MRID 100 wirelessly communicates with console 10 (or with other MRIDs 100 and/or other surgical instruments like grasper 300) so that each MRID may crawl to any suitable location within body cavity “BC” (e.g., adjacent to cannula 200 or spaced far from cannula 200). In particular, MRID 100 can be tele-manipulated so that legs 130 of MRID 100 crawl along tissue “T” to achieve a desired position or be moved to change or achieve a different position. Such process or repositioning can be repeated as desired. Camera 120 of MRID 100 can also be positioned or repositioned as desired to obtain desired imagery or sound for effectuating a procedure (e.g., surgical, therapeutic, and/or diagnostic). When the procedure is completed, MRID 100 can be moved back toward cannula 200 so that grasper 300 can grab MRID 100 and remove MRID 100 through cannula 200 with MRID 100 positioned win the retracted position thereof.

In some embodiments, one or more the presently disclosed feet may include a single arm having hooks or barbs similar to insect feet for grasping tissue like a grappling hook.

As can be appreciated, securement of any of the components of the presently disclosed apparatus can be effectuated using known securement techniques such welding, crimping, gluing, fastening, etc.

Persons skilled in the art will understand that the structures and methods specifically described herein and illustrated in the accompanying figures are non-limiting exemplary embodiments, and that the description, disclosure, and figures should be construed merely as exemplary of particular embodiments. It is to be understood, therefore, that the present disclosure is not limited to the precise embodiments described, and that various other changes and modifications may be effected by one skilled in the art without departing from the scope or spirit of the disclosure. Additionally, it is envisioned that the elements and features illustrated or described in connection with one exemplary embodiment may be combined with the elements and features of another without departing from the scope of the present disclosure, and that such modifications and variations are also intended to be included within the scope of the present disclosure. Indeed, any combination of any of the presently disclosed elements and features is within the scope of the present disclosure. Accordingly, the subject matter of the present disclosure is not to be limited by what has been particularly shown and described.

1. A micro robotic imaging device, comprising:
a housing;
a camera coupled to the housing; and
legs coupled to the housing, the legs movable relative to one another to move the camera within a patient's body for capturing imagery of the patient's body.

2. The micro robotic imaging device of claim 1, wherein the legs, the camera, or combinations thereof are in wireless communication with a remote console.

3. The micro robotic imaging device of claim 2, wherein the remote console is configured to operate the legs, the camera, or combinations thereof.

4. The micro robotic imaging device of claim 1, wherein the camera is movable relative to the housing.

5. The micro robotic imaging device of claim 1, wherein the legs are movable relative to the housing between a retracted position and an extended position.

6. The micro robotic imaging device of claim 5, wherein when the legs are disposed in the retracted position, the micro robotic imaging device is configured to pass through a surgical cannula for selectively positioning the micro robotic imaging device within the patient's body.

7. The micro robotic imaging device of claim 5, wherein when the legs are disposed in the extended position, the legs are movable relative to the housing to enable the micro robotic imaging device to crawl.

8. The micro robotic imaging device of claim 1, wherein a first leg of the legs includes a foot configured to grasp tissue.

9. The micro robotic imaging device of claim 8, wherein the foot is movable relative to the first leg.

10. The micro robotic imaging device of claim 9, wherein the foot includes a first arm and a second arm.

11. A micro robotic imaging system for capturing imagery of a patient's body cavity, the micro robotic imaging system comprising:
a remote console; and
a micro robotic imaging device in communication with the remote console, the micro robotic imaging device including:
a housing;
a camera coupled to the housing; and
legs movably coupled to the housing to enable the micro robotic imaging device to crawl through the patient's body cavity.

12. The micro robotic imaging system of claim 11, wherein the remote console and the micro robotic imaging device are wirelessly coupled.

13. The micro robotic imaging system of claim 12, wherein the remote console is actuatable to operate the micro robotic imaging device.

14. The micro robotic imaging system of claim 11, further comprising a surgical cannula, wherein the micro robotic imaging device is configured to pass through the surgical cannula.

15. The micro robotic imaging system of claim 14, wherein the legs are movable between a retracted position and an extended position, wherein in the retracted position, the micro robotic imaging device can pass through the surgical cannula, and wherein in the extended position, the micro robotic imaging device cannot pass through the surgical cannula.

16. The micro robotic imaging system of claim 15, further comprising a grasper configured to pass the micro robotic imaging device through the surgical cannula and selectively position the micro robotic imaging device within the patient's body cavity.

17. A method of in vivo imaging of a patient's body with a micro robotic imaging system, the method comprising:
advancing a micro robotic imaging device into the patient's body;
remotely controlling the micro robotic imaging device so that the micro robotic imaging device crawls through the patient's body; and
capturing imagery of the patient's body with the micro robotic imaging device.

18. The method of claim 17, further comprising moving legs of the micro robotic imaging device so that the micro robotic imaging device crawls along tissue within the patient's body.

19. The method of claim 17, further comprising moving a camera of the micro robotic imaging device to capture the imagery of the patient's body.

20. The method of claim 18, further comprising moving the legs of the micro robotic imaging device to enable the micro robotic imaging device to fit through a surgical cannula for selective passage into or out of the patient's body, the surgical cannula having a diameter through which the micro robotic imaging device passes, the diameter being about 5 mm to about 15 mm.
